(19) United States
(12) Patent Application Publication
(10) Pub. No.: US 2025/0228765 A1
(43) Pub. Date: Jul. 17, 2025

(54) TREATING KNEE JOINT PAIN BY INJECTING RESINIFERATOXIN AT ULTRA LOW DOSES

(71) Applicant: Gruenenthal GmbH, Aachen (DE)

(72) Inventors: Zouaoui BOUREZG, Aachen (DE); Michael GAUTROIS, Aachen (DE)

(73) Assignee: Gruenenthal GmbH, Aachen (DE)

(21) Appl. No.: 19/022,396

(22) Filed: Jan. 15, 2025

(30) Foreign Application Priority Data
Jan. 15, 2024 (EP) 24151807.5
Jan. 22, 2024 (EP) 24153125.0
Jan. 22, 2024 (EP) 24153126.8
May 13, 2024 (EP) 24175474.6

(51) Int. Cl. A61K 9/00; A61K 31/357; A61K 47/10; A61K 47/18; A61K 47/26; A61P 19/02; A61P 29/02

(52) U.S. Cl. CPC A61K 9/0019; A61K 31/357; A61K 47/10; A61K 47/18; A61K 47/26; A61P 19/02; A61P 29/02

(57) ABSTRACT

The invention relates to a pharmaceutical dosage form for injection, preferably intraarticular injection into a knee, comprising or essentially consisting of (i) Resiniferatoxin; preferably at a dose within the range of from 50 to 900 ng, preferably less than 500 ng; (ii) water; (iii) an organic solvent; preferably ethanol; (iv) a surfactant; preferably D-α-tocopherol polyethylene glycol succinate (TPGS); (v) optionally, a buffer; and (vi) optionally, one or more inorganic salts; wherein the weight content of the water is greater than the weight content of the organic solvent. The invention further relates to a liquid pharmaceutical dosage form comprising Resiniferatoxin at a dose within the range of from 50 to 900 ng, preferably less than 500 ng for use in the treatment of knee joint pain, preferably osteoarthritic knee joint pain, by intraarticular administration, preferably intraarticular injection. The invention further relates to administration regimens for the treatment of knee joint pain, preferably osteoarthritic knee joint pain, by intraarticular administration, preferably intraarticular injection of liquid pharmaceutical dosage forms comprising Resiniferatoxin at a dose within the range of from 50 to 900 ng, preferably less than 500 ng.

Priority is claimed of European Patent Application No. 24 151 807.5, filed Jan. 15, 2024; European Patent Application No. 24 153 125.0, filed Jan. 22, 2024; European Patent Application No. 24 153 126.8, filed Jan. 22, 2024; and European Patent Application No. 24 175 474.6, filed May 13, 2024, the disclosures of which patent applications are hereby incorporated herein by reference.

The invention relates to a pharmaceutical dosage form for injection, preferably intraarticular injection into a knee, comprising or essentially consisting of (i) Resiniferatoxin; preferably at a dose within the range of from 50 to 900 ng, more preferably less than 500 ng; (ii) water; (iii) an organic solvent; preferably ethanol; (iv) a surfactant; preferably D-α-tocopherol polyethylene glycol succinate (TPGS); (v) optionally, a buffer; and (vi) optionally, one or more inorganic salts; wherein the weight content of the water is greater than the weight content of the organic solvent. The invention further relates to a liquid pharmaceutical dosage form comprising Resiniferatoxin at a dose within the range of from 50 to 900 ng, preferably less than 500 ng, for use in the treatment of knee joint pain, preferably osteoarthritic knee joint pain, by intraarticular administration, preferably intraarticular injection. The invention further relates to administration regimens for the treatment of knee joint pain, preferably osteoarthritic knee joint pain, by intraarticular administration, preferably intraarticular injection of liquid pharmaceutical dosage forms comprising Resiniferatoxin at a dose within the range of from 50 to 900 ng, preferably less than 500 ng.

Resiniferatoxin (RTX) is a naturally occurring compound produced by the Moroccan cactus Euphorbia resinfera. Resiniferatoxin is a potent and selective agonist of the transient receptor potential vanilloid 1 (TRPV1) being developed for treatment of pain related to knee osteoarthritis.

Resiniferatoxin-mediated activation of TRPV1-expressing neurons is followed by reversible defunctionalization of the peripheral terminals of C- and A-delta nerve fibers that can lead to prolonged analgesia.

Resiniferatoxin has been suggested for treating various types of pain and various other disorders and conditions by various routes of administration.

C. Sila et al., BJU Int. 2001 September; 88(4):355-60. doi: 10.1046/j.1464-410x.2001.02339.x. report that seven patients with detrusor hyperreflexia were treated intravesically with Resiniferatoxin dissolved in 10% ethanol in saline (50 nmol/L solution in two and 100 nmol/L in five).

H. Kuo, J Urol. 2003 September; 170(3):835-9. doi: 10.1097/01.ju.0000081652.31524.27 discloses intravesical Resiniferatoxin therapy with 10 ml of 100 nM Resiniferatoxin in 10% ethanol solution for 40 minutes.

M. de Sèze et al., J Urol. 2004 January; 171(1):251-5. doi: 10.1097/01.ju.0000100385.93801.d4 relates to a comparison of the efficacy and tolerability of nonalcohol capsaicin vs. Resiniferatoxin in 10% alcohol in neurogenic patients with detrusor hyperreflexia.

C. Silva et al., Eur Urol. 2005 October; 48(4):650-5. doi: 10.1016/j.eururo.2005.04.012 relates to an investigation wherein patients with spinal neurogenic detrusor overactivity were randomized to receive intravesically 50 nM Resiniferatoxin dissolved in 10% ethanol in saline.

E. Y. Kissin et al., Anesth Analg. 2005 November; 101(5):1433-1439. doi: 10.1213/01.ANE. 0000180998.29890.B0. relates to the effects of intraarticular Resiniferatoxin in experimental knee-joint arthritis.

J. C. Shin et al., Spinal Cord. 2006 May; 44(5):309-14. doi: 10.1038/sj.sc.3101851 relates to an investigation wherein 100 ml of Resiniferatoxin solution, at a concentration of 100 nM diluted in 10% ethanol, was intravesically instilled into the bladder of 15 spinal cord injury patients with neurogenic detrusor overactivity.

C. Silva et al., BMC Urol. 2007 Jun. 11:7:9. doi: 10.1186/1471-2490-7-9 relates to an investigation wherein patients with overactive bladder were instilled with 100 ml of 50 nM Resiniferatoxin in 10% ethanol in saline.

I. Vetter et al., Eur J Pain. 2008 May; 12(4):441-54. doi: 10.1016/j.ejpain.2007.07.001 report that ethanol can sensitize TRPV1-mediated responses, but the pathways contributing to the potentiation of TRPV1 by ethanol have not been clearly defined. The presented results suggest that morphine may be of limited use in inhibiting nociceptive TRPV1 responses that have been sensitized by exposure to ethanol.

I. Kissin et al., Curr Top Med Chem. 2011; 11(17):2159-70. doi: 10.2174/156802611796904924 reviews therapeutic targeting of TRPV1 by Resiniferatoxin, from preclinical studies to clinical trials.

M. J. Iadarola et al., Resiniferatoxin for Pain Treatment: An Interventional Approach to Personalized Pain Medicine, Open Pain J. 2013; 6: 95-107 reviews preclinical and clinical studies related to Resiniferatoxin. Pain conditions that may be susceptible to treatment with local injection or topical Resiniferatoxin include osteoarthritis, whereas Resiniferatoxin is directly injected into the joint. No dosages are mentioned.

Y. Kim et al., The effects of intra-articular resiniferatoxin on monosodium iodoacetate-induced osteoarthritic pain in rats, Korean J Physiol Pharmacol 2016; 20(1):129-136 teaches that based upon preclinical studies in rats, local delivery of Resiniferatoxin may be beneficial to osteoarthritis patients. However, the major problem with Resiniferatoxin treatment is acute toxicity. No dosages for treating humans are mentioned.

H. Aizawa et al., Int J Urol. 2016 Nov.; 23(11):952-956. doi: 10.1111/iju.13181 relates to an examination whether systemic Resiniferatoxin treatment can desensitize the single-unit afferent activities of Aδ- and C-fibers in rat primary bladder mechanosensitive afferent nerves. Resiniferatoxin (0.3 mg/kg) or its vehicle (10% ethanol) was injected subcutaneously to female Sprague-Dawley rats after the first eye-wipe behavior test with capsaicin.

M. J. Iadarola et al., Long-term pain relief in canine osteoarthritis by a single intra-articular injection of resiniferatoxin, a potent TRPV1 agonist, Pain. 2018 October; 159(10): 2105-2114 document a significant and prolonged analgesic effect of a single intraarticular injection of 10 μg of RTX in naturally-occurring canine osteoarthritis, which is physiologically, and symptomatically analogous to human osteoarthritis. 7 dogs with osteoarthritis were treated with a single intraarticular injection of 10 μg of Resiniferatoxin. Dose estimations, and other critical information from previous canine studies translated directly to the human cancer pain trial. This pilot study is said to provide proof-of-concept data for translation to a human osteoarthritis trial. No dosages for treating humans are quantified.

D. Leiman et al., abstract 197, Osteoarthritis and Cartilage 28 (2020) S86-S527 report about preliminary results from a phase 1b double-blind study to assess the safety, tolerability and efficacy of intraarticular Resiniferatoxin or placebo for the treatment of moderate to severe pain due to osteoarthritis of the knee. In this multicenter, randomized, double-blind, placebo-controlled study, eligible subjects aged 35-85 years, with baseline pain score in the index knee of ≥5 but 9 (WOMAC pain subscale question A1) were treated with a one-time intra-articular dose of Resiniferatoxin at escalating dose level cohorts of 5 μg, 12.5 μg, 20 μg and 30 μg in 5 or 10 mL of saline, or saline placebo. Resiniferatoxin demonstrated safety when given as onetime intraarticular injection up to the planned highest dose of 30 μg. Preliminary efficacy was also observed. Anecdotal long-term follow up suggests sustained pain relief beyond day 365.

A. Szallasi, Resiniferatoxin: Nature's Precision Medicine to Silence TRPV1-Positive Afferents, Int. J. Mol. Sci. 2023, 24, 15042 reviews medical utility of Resiniferatoxin. A breakthrough therapy designation for intraarticular Resiniferatoxin at a dose of 12.5 μg to treat pin associated with knee osteoarthritis is discussed (cf. s. Lopez et al., J. Analgesics of the Future: IA Resiniferatoxin for Osteoarthritic Knee Pain. PractPainManag. 2022; 22(2), updated Mar. 1, 2022).

US 2001 0006982 A1 (WO 1999/009970 A1) relates to a method of treating neurogenic urinary dysfunction that comprises contacting urinary bladder mucosa of a patient afflicted with neurogenic urinary dysfunction with an effective dose of a homovanilloid compound, in particular a compound selected from the group RTX, TYX, 20-homovanillyl-mezerein or 20-homovanillyl-12-deoxyphorbol-13-phenylacetate.

US 2004 0146590 A1, US 2010 0222385 A1, US 2013 0210905 A1, US 2015 0051271 A1, and US 2018 0117004 A1 provide methods and kits for the selective ablation of pain-sensing neurons. The methods comprise administration of a vanilloid receptor agonist to a ganglion in an amount that causes death of vanilloid receptor-bearing neurons.

US 2004 0161481 A1 (WO 2004/056305 A2) provides compositions and methods for relieving pain at a site in a human or animal in need thereof by administering at a discrete site in a human or animal in need thereof a dose of capsaicin in an amount effective to denervate a discrete site without eliciting an effect outside the discrete location, the dose of capsaicin ranging from 1 μg to 3000 μg.

US 2005 0019258 A1 (WO 2005/004883 A2) relates to a method for the evaluation of potential therapeutic agents for the treatment of incontinence, as well as to a method for discovering new therapeutic agents for the treatment of incontinence.

US 2005 0019436 A1 (WO 2004/058286 A1) and US 2007 0036876 A1 (WO 2004/056305A2) disclose a method for attenuating pain at a site in a human or animal in need thereof, comprising: administering at a discrete painful site in a human or animal in need thereof a single injectable or implantable dose of a capsaicinoid in an amount effective to denervate said discrete site without eliciting an effect outside the discrete location and to attenuate pain emanating from said site, said effective dose being from about 1 μg to about 5000 μg of capsaicin or a therapeutically equivalent dose of a capsaicinoid other than capsaicin.

US 2006 0269628 A1 (WO 2004/058286 A1) provides compositions and methods for attenuating or relieving pain at a site in a human or animal in need thereof by infiltrating at a surgical site or open wound in a human or animal a dose of capsaicinoid in an amount effective to denervate the surgical site or open wound substantially without eliciting an effect outside the surgical site or open wound.

WO 2008/109026 A1 relates to compositions and methods for the treatment of neuropathic or neurogenic pain and/or disorders at least partially associated with neurogenic dysfunction comprising the use of a pharmaceutical composition comprising at least one homovanilloid compound and at least one permeation enhancer.

US 2008 0139641 A1 (WO 2006/069451 A1) relates to a formulation of Resiniferatoxin for the preparation of an agent for the treatment of different pain conditions.

US 2008 0260791 A1 (WO 2004/058286 A1) relates to an injectable or implantable pharmaceutical composition for attenuating pain at a site in a human or animal in need thereof, consisting essentially of a capsaicinoid selected from the group consisting of from 1 μg to 5000 μg of capsaicin, a therapeutically equivalent amount of one or more other capsaicinoids, and combinations thereof; in a pharmaceutically acceptable vehicle for injection or implantation.

US 2009 0209633 A1 (WO 2008/011532 A2) relates to a method of treating inflammatory pain conditions that involves administering an effective amount of a TRPV1 agonist, such as Resiniferatoxin, Tinyatoxin and related potent agonists and their analogs, to a patient to selectively induce nerve terminal depolarization block and/or nerve terminal death in select TRPV1-containing neurons, to provide the desired pain relief without significant permanent damage to cell bodies of the select TRPV-1 containing neurons.

US 2010 047181 A1 (WO 2006 066419 A1) relates to a mixture comprising a vanilloid receptor agonist and a substance inhibiting nerve regeneration. Said mixture is suitable as a painkiller.

US 2010 0137344 A1 (WO 2008/015403 A1) relates to agents which are capable of inducing analgesia in chronic neuropathic pain, associated methods and uses thereof, in particular compounds capable of activating the TRPM8 receptor for the treatment of chronic neuropathic pain.

US 2014 0142073 A1 (WO 2014/075084A2) and US 2015 0133561 A1 (WO 2015/073577 A1) disclose capsaicinoid formulations and methods of treatment which can be utilized to treat/attenuate pain in mammals. Typically, administration is via injection at a discrete site to provide pain relief for an extended period of time. The formulations are administered in a pharmaceutically acceptable vehicle.

The aqueous pharmaceutical compositions utilize pharmaceutically acceptable delivery vehicles that are single phase aqueous/water systems composed of pharmaceutically acceptable solvents including polyethylene glycol, ethanol and a non-ionic surfactant, e.g. polysorbate 80 (PS 80); buffers; NaCl and/or sugar to control osmotic pressure gradients; and hyaluronic acid to control the viscosity of the formulation and aid in formulation stability.

US 2014 0187619 A1 (WO 2013/012892 A2) relates to methods of treatment in subjects suffering from diabetes mellitus or obesity. The methods comprise the step of administering an active agent directly to the small intestine in the subject. The active agents include analgesic agents and, in particular, antinociceptive agents such as capsaicin, Resiniferatoxin, and their analogues.

US 2015 0080460 A1 relates to a method for administration of a formulation of Resiniferatoxin (RTX) directly onto the epicardium, comprising applying a formulation of RTX directly onto the epicardium under the pericardial sac.

US 2015 0190509 A1 (WO 2014/019095 A1) relates to a Resiniferatoxin solution having an enhanced storage stability, in which the Resiniferatoxin is dissolved in a body-compatible solvent which contains a protective gas in solution, wherein the amount of the protective gas is at least 1 wt.-% of the saturation amount of the protective gas in the solvent at ambient temperature and normal pressure. The solution can be used in particular for the treatment of intra-articular pain.

US 2019 0076396 A1 (WO 2019/049112 A1) relates to formulations of Resiniferatoxin (RTX) for intrathecal, intraganglionic intraarticular and pericardial administration, more specifically alcohol-free formulations of RTX comprising a solubilizing component, a monosaccharide or sugar alcohol, a saline buffer, and RTX, and having narrow ranges for pH range and specific gravity. It is alleged that for intraarticular administration, a typical volume injected into an adult knee would be from 3 ml to 10 ml, delivering a total amount of RTX from 5 ng to 50 μg, and that often the amount administered would be from 200 ng to 10 g.

US 2019 0321493 A1 (WO 2018/094262 A2) provides materials and methods for treating regional pain. For example, compositions including one or more analgesics can be selectively administered (e.g., by image-guided injection) to one or more nerves to treat a mammal having regional pain.

WO 2020 132553A1 discloses methods of administering resiniferatoxin (RTX) perineurally for treatment of maladaptive pain, and compositions for use in such methods.

US 2020 0261409 A1 (WO 2017/087803 A1) discloses methods for treating pain using Resiniferatoxin. The Resiniferatoxin may be administered to certain anatomic sites using image-guided delivery. The Resiniferatoxin may be administered in low doses in the range of 0.5 to 3.0 μg of Resiniferatoxin in a human patient.

US 2020 0323800 A1 (WO 2015/160941 A1) relates to TRPV1 selective agonist compositions including a capsaicinoid, a surfactant and an extended release agent, and to methods of manufacture and methods of providing pain relief as well as treating a variety of disorders with such compositions.

US 2021 0007998 A1 relates to nanoparticles comprising Resiniferatoxin (RTX) encapsulated in a poly(lactic-co-glycolic acid) (PLGA) polymer and compositions, especially topical compositions, comprising the nanoparticles.

US 2021 0299090 A1 (WO 2020 139797A1) discloses a method for treating Parkinson's Disease (PD) comprising administering an effective amount of Resiniferatoxin (RTX) by an intrathecal or intracisternal administration.

US 2021 0393515 A1 relates to methods of administering Resiniferatoxin (RTX) perineurally for treatment of maladaptive pain, and compositions for use in such methods.

US 2022 0008384 A1 (WO 2020/191506 A1) relates to a composition comprising capsaicin or a capsaicinoid is used in a method for postoperative pain control. The composition is administered to a site intended for surgery in a patient at least one day before surgery is actually performed.

US 2022 0096428 A1 (WO 2020/154261 A1) relates to methods of administering Resiniferatoxin (RTX) for treatment of osteoarthritis (OA) pain, and compositions for use in such methods.

US 2023 0051321 A1 (WO 2021/209450 A1) relates to non-aqueous solid and liquid compositions comprising Resiniferatoxin and a surfactant. The non-aqueous solid and liquid compositions may be used to prepare aqueous compositions that are used in the treatment of pain, specifically osteoarthritis-related joint pain.

US 2023 0143545 A1 (WO 2021/202084 A1) relates to a method for treating pulmonary inflammatory disease comprising administering an effective amount of Resiniferatoxin (RTX) by an epidural, peri-ganglionic or intra-ganglionic administration. In some embodiments, the dose of RTX for an adult human is from about 0.1 μg to about 100 μg.

US 2023 0270713 A1 (WO 2021/257956 A1) relates to methods of administering Resiniferatoxin (RTX) intravesically for treatment of bladder pain, and compositions for use in such methods.

Further, Resiniferatoxin has been suggested for treating joint pain such as pain due to osteoarthritis of the knee. While it is alleged in US 2019 0076396 A1 (WO 2019/049112 A1) that for intraarticular administration, a typical volume injected into an adult knee would be from 3 ml to 10 ml, delivering a total amount of RTX from 5 ng to 50 μg, and that often the amount administered would be from 200 ng to 10 μg, the clinically tested doses of Resiniferatoxin in fact were in the μg scale, typically 3 μg, 6 μg, 12.5 μg, 20 μg, or 30 μg.

A completed clinical trial (www.clinicaltrials.gov, identifier NCT03542838) relates to a study of Resiniferatoxin for knee pain in moderate to severe osteoarthritis. According to the study plan, Resiniferatoxin was administered as a one-time dose, intraarticularly at a dose level of 5 μg, 12.5 μg, 20 μg, 25 μg, or 30 μg. The study was completed on Nov. 2, 2021 and no results have been posted.

A withdrawn clinical trial (www.clinicaltrials.gov, identifier NCT04044742) was planned as a phase 3 study to evaluate the efficacy and safety of Resiniferatoxin for pain due to osteoarthritis of the knee. According to the study plan, 12.5 μg of Resiniferatoxin in 5 ml volume should be administered intraarticularly as a one-time dose.

A withdrawn clinical trial (www.clinicaltrials.gov, identifier NCT04386980) was planned to evaluate resiniferatoxin in patients with knee osteoarthritis whose total knee replacement surgery is delayed. According to the study plan, 12.5 μg of Resiniferatoxin in 5 ml volume should be administered once intraarticularly.

An active, not recruiting clinical trial (www.clinicaltrials.gov, identifier NCT04885972) is planned as a study to evaluate intraarticular Resiniferatoxin to treat moderate to severe pain from knee osteoarthritis. According to the study plan, 7.5 μg, 10 μg, 12.5 μg, 15 μg, or 20 μg in 5 ml should be injected once intraarticularly.

With press release of Sep. 7, 2023, Sorrento Therapeutics announced positive phase 2a clinical trial results for Resiniferatoxin for the treatment of knee pain in moderate to severe osteoarthritis of the knee patients. The administered doses of Resiniferatoxin amounted to 7.5 to 20 μg.

The known pharmaceutical dosage forms of Resiniferatoxin of the prior art for treating knee joint pain, especially osteoarthritic knee joint pain, are not satisfactory in every respect and there is a demand for improved dosage forms, especially for enhanced tissue distribution.

It is an object of the invention to provide dosage forms of Resiniferatoxin that are useful for treating joint pain, preferably knee joint pain due to osteoarthritis, and that have advantages compared to the prior art, especially improved distribution within the knee to enhance efficacy. It is an object of the invention to provide relief of knee joint pain, especially osteoarthritic knee joint pain. In particular, it is an object of the invention to provide methods and medicaments for treating knee joint pain, especially osteoarthritic knee joint pain, that have advantages compared to the prior art.

This object has been achieved by the subject-matter of the patent claims.

It has been surprisingly found that Resiniferatoxin provides relief of knee joint pain, especially osteoarthritic knee joint pain, when it is intraarticularly administered at significantly lower doses than suggested in the prior art. While it has been suggested in the prior art to treat osteoarthritic knee joint pain by intraarticularly administering doses of Resiniferatoxin on the μg scale, e.g. 12.5 μg, it has now been surprisingly found that a much lower dose of Resiniferatoxin amounting to only 400 ng or even only 100 ng provides satisfactory analgesia, especially when the administered dosage form contains aqueous ethanol. It can be expected that such lower doses provide better tolerability and safety than the conventional doses of Resiniferatoxin on the μg scale.

Further, it has been surprisingly found that even such ultra low doses of Resiniferatoxin provide prolonged pain relief of several months after intraarticular administration. Thus, the ultra low doses according to the invention appear particularly useful for long term treatment wherein the patient receives repeated intraarticular administration over several months or years.

Still further, it has been surprisingly found that upon intraarticular administration the distribution of Resiniferatoxin within the knee can be improved when the injection formulation contains an organic solvent, especially ethanol.

### SUMMARY OF THE INVENTION

A first aspect of the invention relates to a pharmaceutical dosage form for injection, preferably intraarticular injection into a knee, comprising or essentially consisting of
(i) Resiniferatoxin; preferably at a dose within the range of from 50 to 900 ng, more preferably less than 500 ng;
(ii) water;
(iii) an organic solvent; preferably ethanol;
(iv) a surfactant; preferably D-α-tocopherol polyethylene glycol succinate (TPGS);
(v) optionally, a buffer; and
(vi) optionally, one or more inorganic salts;
wherein the weight content of the water is greater than the weight content of the organic solvent.

### BRIEF DESCRIPTION OF THE DRAWINGS

The invention will now be described in greater detail with reference to the drawings, wherein:

FIG. 1 shows the efficacy in terms of visual analog scale pain in motion as changed over time;

FIG. 2 shows the PK concentration time plot for inventive example I-1;

FIG. 3 shows the PK concentration time plot for comparative example C-1;

FIG. 4 shows the PK concentration time plot for comparative example C-2;

FIG. 5 shows PK in synovial fluid; and

FIG. 6 shows PK in plasma.

The pharmaceutical dosage form for injection is typically a liquid pharmaceutical dosage form.

Another aspect of the invention relates to a liquid pharmaceutical dosage form comprising Resiniferatoxin at a dose within the range of from 50 to 900 ng for use in the treatment of knee joint pain, preferably osteoarthritic knee joint pain, by intraarticular administration, preferably intraarticular injection.

A further aspect of the invention relates to a method of treating knee joint pain, preferably osteoarthritic knee joint pain, comprising intraarticularly administering, preferably intraarticularly injecting, a liquid pharmaceutical dosage form comprising Resiniferatoxin at a dose within the range of from 50 to 900 ng, preferably less than 500 ng to a subject in need thereof.

Still another aspect of the invention relates to the use of Resiniferatoxin for the preparation of a liquid pharmaceutical dosage form comprising Resiniferatoxin at a dose within the range of from 50 to 900 ng, preferably less than 500 ng for the treatment of knee joint pain, preferably osteoarthritic knee joint pain, by intraarticular administration, preferably intraarticular injection.

For the purpose of the specification, a “pharmaceutical dosage form” is an administration unit that contains a defined single dose of Resiniferatoxin that is to be administered to a patient in need thereof, preferably a human patient, all at once.

For the purpose of the specification, “essentially free” means that such component, if present at all, is present in an amount not materially affecting the essential characteristics of the mixture.

Preferably, the content of such component relative to the total weight of the mixture is at most 1.0 wt.-%, more preferably at most 0.5 wt.-%, still more preferably at most 0.1 wt.-%, most preferably at most 0.001 wt.-%, and in particular below the analytical detection limit.

For the purpose of the specification, “essentially consisting of” means that specific further components can be present, namely those not materially affecting the essential characteristics of the mixture. Preferably, the total content of such further components relative to the total weight of the mixture is at most 1.0 wt.-%, more preferably at most 0.5 wt.-%, still more preferably at most 0.1 wt.-%, most preferably at most 0.001 wt.-%, and in particular below the analytical detection limit.

The pharmaceutical dosage form according to the invention is preferably a liquid, preferably a solution.

The pharmaceutical dosage form according to the invention is useful and devoted for injection, preferably for intraarticular injection, more preferably for intraarticular injection into the knee.

The pharmaceutical dosage form according to the invention contains Resiniferatoxin.

Preferably, Resiniferatoxin is the only pharmacologically active ingredient that is contained in the pharmaceutical dosage form.

The liquid dosage form according to the invention is for use in the treatment of knee joint pain, preferably osteoarthritic knee joint pain (knee joint pain due to osteoarthritis). Thus, the pain to be treated is preferably associated to osteoarthritis of the knee.

The liquid pharmaceutical dosage form according to the invention comprises Resiniferatoxin at a dose within the range of from 50 to 900 ng, preferably less than 500 ng.

Preferably, the administered dose of Resiniferatoxin within this range provides analgesia for at least 1 month post administration; preferably at least 2 months, more preferably at least 3 months, still more preferably at least 4 months, yet more preferably at least 5 months, even more preferably at least 6 months, most preferably at least 7 months, and in particular at least 8 months.

Resiniferatoxin (RTX, C₃₇H₄₀O₉, Mr 628.718 g/mol, CAS 57444-62-9, DrugBank Accession No DB06515) is the compound of the following structure:

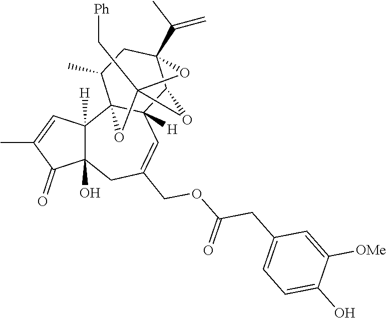 [SMILES: C=C(C)[C@]12C[C@@H](C)[C@@]34OC(Cc5ccccc5)(O[C@@H]1[C@@H]3C=C(COC(=O)Cc1ccc(O)c(OC)c1)C[C@]1(O)C(=O)C(C)=C[C@@H]41)O2]

and has the systematic name 4-hydroxy-3-methoxy-benzeneacetic acid, [(2S,3aR,3bS,6aR,9aR,9bR, 10R,11aR)-3a, 3b, 6,6a, 9a,10,11,11a-octahydro-6a-hydroxy-8,10-dimethyl-11a-(1-methylethenyl)-7-oxo-2-(phenylmethyl)-7H-2,9b-epoxyazuleno[5,4-e]-1,3-benzodioxol-5-yl]methyl ester. It is commercially available.

Preferably, Resiniferatoxin is completely dissolved in the dosage form being a liquid.

Preferably, the dose of Resiniferatoxin is less than 3.2 μg.

Preferably, the dose of Resiniferatoxin is within the range of from 50 to 900 ng; preferably less than 800 ng, more preferably less than 700 ng, still more preferably less than 600 ng, yet more preferably less than 500 ng.

Preferably, the dose of Resiniferatoxin is at most 850 ng; preferably at most 800 ng, more preferably at most 750 ng, still more preferably at most 700 ng, yet more preferably at most 650 ng, even more preferably at most 600 ng, most preferably at most 550 ng, and in particular at most 500 ng.

Preferably, the dose of Resiniferatoxin is at most 490 ng; preferably at most 480 ng, more preferably at most 470 ng, still more preferably at most 460 ng, yet more preferably at most 450 ng, even more preferably at most 440 ng, most preferably at most 430 ng, and in particular at most 420 ng.

Preferably, the dose of Resiniferatoxin is at least 100 ng; preferably at least 150 ng, more preferably at least 200 ng, still more preferably at least 250 ng, yet more preferably at least 300 ng, even more preferably at least 350 ng, most preferably at least 400 ng, and in particular at least 450 ng.

In preferred embodiments, the dose of Resiniferatoxin is within the range of 100±90 ng; preferably 100±80 ng, more preferably 100±70 ng, still more preferably 100±60 ng, yet more preferably 100±50 ng, even more preferably 100±40 ng, most preferably 100±30 ng, and in particular 100±20 ng.

In preferred embodiments, the dose of Resiniferatoxin is within the range of 200±180 ng; preferably 200±160 ng, more preferably 200±140 ng, still more preferably 200±120 ng, yet more preferably 200±100 ng, even more preferably 200±80 ng, most preferably 200±60 ng, and in particular 200±40 ng.

In preferred embodiments, the dose of Resiniferatoxin is within the range of 300±200 ng; preferably 300±175 ng, more preferably 300±150 ng, still more preferably 300±125 ng, yet more preferably 300±100 ng, even more preferably 300±75 ng, most preferably 300±50 ng, and in particular 300±25 ng.

In preferred embodiments, the dose of Resiniferatoxin is within the range of 400±200 ng; preferably 400±175 ng, more preferably 400±150 ng, still more preferably 400±125 ng, yet more preferably 400±100 ng, even more preferably 400±75 ng, most preferably 400±50 ng, and in particular 400±25 ng. A dose of about 400 ng is most preferred.

In preferred embodiments, the dose of Resiniferatoxin is within the range of 500±200 ng; preferably 500±175 ng, more preferably 500±150 ng, still more preferably 500±125 ng, yet more preferably 500±100 ng, even more preferably 500±75 ng, most preferably 500±50 ng, and in particular 500±25 ng.

Preferably, the weight concentration of Resiniferatoxin is at least 5.0 μg/l; preferably at least 10 μg/l, more preferably at least 20 μg/l, still more preferably at least 30 μg/l, yet more preferably at least 40 μg/l, even more preferably at least 50 μg/l, most preferably at least 60 μg/l, and in particular at least 70 μg/l.

Preferably, the weight concentration of Resiniferatoxin is at most 250 μg/l; preferably at most 225 μg/l, more preferably at most 200 μg/l, still more preferably at most 175 μg/l, yet more preferably at most 150 μg/l, even more preferably at most 125 μg/l, most preferably at most 100 μg/l, and in particular at most 90 μg/l.

Preferably, the weight concentration of Resiniferatoxin is within the range of 80.0±20.0 μg/l; preferably 80.0±17.5 μg/l, more preferably 80.0±15.0 μg/l, still more preferably 80.0±12.5 μg/l, yet more preferably 80.0±10.0 μg/l, even more preferably 80.0±7.5 μg/l, most preferably 80.0±5.0 μg/l, and in particular 80.0±2.5 μg/l.

Preferably, the dosage form according to the invention has a total volume of at least 1.0 ml; preferably at least 1.5 ml, more preferably at least 2.0 ml, still more preferably at least 2.5 ml, yet more preferably at least 3.0 ml, even more preferably at least 3.5 ml, most preferably at least 4.0 ml, and in particular at least 4.5 ml.

Preferably, the dosage form according to the invention has a total volume of at most 13 ml; preferably at most 12 ml, more preferably at most 11 ml, still more preferably at most 10 ml, yet more preferably at most 9.0 ml, even more preferably at most 8.0 ml, most preferably at most 7.0 ml, and in particular at most 6.0 ml.

Preferably, the dosage form according to the invention has a total volume within the range of from 1.0 to 7.5 ml.

In preferred embodiments, the dosage form according to the invention has a total volume within the range of 3.00±2.00 ml; preferably 3.00±1.75 ml, more preferably 3.00±1.50 ml, still more preferably 3.00±1.25 ml, yet more preferably 3.00±1.00 ml, even more preferably 3.00±0.75 ml, most preferably 3.00±0.50 ml, and in particular 3.00±0.25 ml.

In further preferred embodiments, the dosage form according to the invention has a total volume within the range of 4.00±2.00 ml; preferably 4.00±1.75 ml, more preferably 4.00±1.50 ml, still more preferably 4.00±1.25 ml, yet more preferably 4.00±1.00 ml, even more preferably 4.00±0.75 ml, most preferably 4.00±0.50 ml, and in particular 4.00±0.25 ml.

In most preferred embodiments, the dosage form according to the invention has a total volume within the range of 5.00±2.00 ml; preferably 5.00±1.75 ml, more preferably 5.00±1.50 ml, still more preferably 5.00±1.25 ml, yet more preferably 5.00±1.00 ml, even more preferably 5.00±0.75 ml, most preferably 5.00±0.50 ml, and in particular 5.00±0.25 ml.

In further preferred embodiments, the dosage form according to the invention has a total volume within the range of 6.00±2.00 ml; preferably 6.00±1.75 ml, more preferably 6.00±1.50 ml, still more preferably 6.00±1.25 ml, yet more preferably 6.00±1.00 ml, even more preferably 6.00±0.75 ml, most preferably 6.00±0.50 ml, and in particular 6.00±0.25 ml.

In additional preferred embodiments, the dosage form according to the invention has a total volume within the range of 7.00±2.00 ml; preferably 7.00±1.75 ml, more preferably 7.00±1.50 ml, still more preferably 7.00±1.25 ml, yet more preferably 7.00±1.00 ml, even more preferably 7.00±0.75 ml, most preferably 7.00±0.50 ml, and in particular 7.00±0.25 ml.

Preferably, the dosage form has a weight concentration of Resiniferatoxin within the range of 80.0±20.0 μg/l; preferably 80.0±17.5 μg/l, more preferably 80.0±15.0 μg/l, still more preferably 80.0±12.5 μg/l, yet more preferably 80.0±10.0 μg/l, even more preferably 80.0±7.5 μg/l, most preferably 80.0±5.0 μg/l, and in particular 80.0±2.5 μg/l.

In particularly preferred embodiments, the dose of Resiniferatoxin is 400 ng (0.4 μg) in a volume of 5.00 ml (0.005 l) such that the weight concentration of Resiniferatoxin is 80 μg/l corresponding to a molar concentration of about 127 nM.

Preferably, Resiniferatoxin is completely dissolved in the liquid.

Preferably, the dosage form according to the invention contains water, i.e. is aqueous.

Preferably, the weight content of water is at least 88 wt.-%; preferably at least 89 wt.-%, more preferably at least 90 wt.-%, still more preferably at least 91 wt.-%, yet more preferably at least 92 wt.-%, even more preferably at least 93 wt.-%, most preferably at least 94 wt.-%, and in particular preferably at least 95 wt.-%, in each case relative to the total weight of the dosage form.

The pharmaceutical dosage form according to the invention contains an organic solvent.

Preferably, the organic solvent is ethanol or dimethyl sulfoxide.

Preferably, the organic solvent is ethanol, i.e. the dosage form according to the invention contains ethanol, i.e. is ethanolic. The invention preferably combines the advantages of administering Resiniferatoxin at very low dosages with the advantages of ethanol.

Preferably, the amount of the organic solvent, preferably ethanol, is at least 10 mg; preferably at least 25 mg, more preferably at least 50 mg, still more preferably at least 75 mg, yet more preferably at least 100 mg, even more preferably at least 125 mg, most preferably at least 150 mg, and in particular at least 175 mg.

Preferably, the amount of the organic solvent, preferably ethanol, is at most 375 mg; preferably at most 350 mg, more preferably at most 325 mg, still more preferably at most 300 mg, yet more preferably at most 275 mg, even more preferably at most 250 mg, most preferably at most 225 mg, and in particular at most 200 mg.

Preferably, the amount of the organic solvent, preferably ethanol, is within the range of 200±80 mg; preferably 200±70 mg, more preferably 200±60 mg, still more preferably 200±50 mg, yet more preferably 200±40 mg, even more preferably 200±30 mg, most preferably 200±20 mg, and in particular 200±10 mg.

Preferably, the volume content of the organic solvent, preferably ethanol, is at least 1.0 vol.-%; preferably at least 1.5 vol.-%, more preferably at least 2.0 vol.-%, still more preferably at least 2.5 vol.-%, yet more preferably at least 3.0 vol.-%, even more preferably at least 3.5 vol.-%, most preferably at least 4.0 vol.-%, and in particular at least 4.5 vol.-%, in each case relative to the total volume of the dosage form.

Preferably, the volume content of the organic solvent, preferably ethanol, is at most 10.0 vol.-%; preferably at most 9.0 vol.-%, more preferably at most 8.0 vol.-%, still more preferably at most 7.5 vol.-%, yet more preferably at most 7.0 vol.-%, even more preferably at most 6.5 vol.-%, most preferably at most 6.0 vol.-%, and in particular at most 5.5 vol.-%, in each case relative to the total volume of the dosage form.

Preferably, the volume content of the organic solvent, preferably ethanol, is within the range of 5.0±4.0 vol.-%; preferably 5.0±3.5 vol.-%, more preferably 5.0±3.0 vol.-%, still more preferably 5.0±2.5 vol.-%, yet more preferably 5.0±2.0 vol.-%, even more preferably 5.0±1.5 vol.-%, most preferably 5.0±1.0 vol.-%, and in particular 5.0±0.5 vol.-%, in each case relative to the total volume of the dosage form.

Preferably, the weight content of the organic solvent, preferably ethanol, is at least 2.00 wt.-%; preferably at least 2.25 wt.-%, more preferably at least 2.50 wt.-%, still more preferably at least 2.75 wt.-%, yet more preferably at least 3.00 wt.-%, even more preferably at least 3.25 wt.-%, most preferably at least 3.50 wt.-%, and in particular at least 3.75 wt.-%, in each case relative to the total weight of the dosage form.

Preferably, the weight content of the organic solvent, preferably ethanol, is at most 6.00 wt.-%; preferably at most 5.75 wt.-%, more preferably at most 5.50 wt.-%, still more preferably at most 5.25 wt.-%, yet more preferably at most 5.00 wt.-%, even more preferably at most 4.75 wt.-%, most preferably at most 4.50 wt.-%, and in particular at most 4.25 wt.-%, in each case relative to the total weight of the dosage form.

Preferably, the weight content of the organic solvent, preferably ethanol, is within the range of 4.0±3.9 wt.-%; preferably 4.0±3.5 wt.-%, more preferably 4.0±3.0 wt.-%, still more preferably 4.0±2.5 wt.-%, yet more preferably 4.0±2.0 wt.-%, even more preferably 4.0±1.5 wt.-%, most preferably 4.0±1.0 wt.-%, and in particular 4.0±0.5 wt.-%, in each case relative to the total weight of the dosage form.

Preferably, the dosage form according to the invention is an aqueous ethanolic solution.

Preferably, the volume concentration of ethanol is within the range of 5.0±4.0 vol.-%; preferably 5.0±3.5 vol.-%, more preferably 5.0±3.0 vol.-%, still more preferably 5.0±2.5 vol.-%, yet more preferably 5.0±2.0 vol.-%, even more preferably 5.0±1.5 vol.-%, most preferably 5.0±1.0 vol.-%, and in particular 5.0±0.5 vol.-%, relative to the total volume of the dosage form.

The pharmaceutical dosage form according to the invention contains a surfactant.

Preferably, the surfactant is a nonionic surfactant.

Preferably, the surfactant in its neat state is a solid at 25° C.

In preferred embodiments, the surfactant is D-α-tocopherol polyethylene glycol succinate (TPGS); preferably selected from TPGS 200, TPGS 238, TPGS 400, TPGS 600, TPGS 1000, TPGS 2000, TPGS 3400, TPGS 3500, TPGS 4000, TPGS 6000, and mixtures thereof, wherein the numbers indicate the molecular weight of the polyethylene glycol chain. Preferred is INN Tocophersolan, i.e. a water-soluble amphipathic formulation of d-alpha-tocopherol succinate coupled, through a succinate linker, to polyethylene glycol (PEG) 1000. Thus, preferably, the surfactant is D-α-tocopherol polyethylene glycol 1000 succinate (TPGS 1000, CAS 9002-96-4) which has a molecular weight of about 1500 g/mol.

In other preferred embodiments, the surfactant is selected from polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), and polysorbate 80 (polyoxyethylene (20) sorbitan monooleate); preferably polysorbate 80 (e.g. Tween® 80).

Preferably, the amount of the surfactant, preferably TPGS, is at least 2.0 mg; preferably at least 4.0 mg, more preferably at least 6.0 mg, still more preferably at least 8.0 mg, yet more preferably at least 10.0 mg, even more preferably at least 12.0 mg, most preferably at least 14.0 mg, and in particular at least 16.0 mg.

Preferably, the amount of the surfactant, preferably TPGS, is at most 55 mg; preferably at most 50 mg, more preferably at most 45 mg, still more preferably at most 40 mg, yet more preferably at most 35 mg, even more preferably at most 30 mg, most preferably at most 25 mg, and in particular at most 20 mg.

Preferably, the amount of the surfactant, preferably TPGS, is within the range of 17.5±16.0 mg; preferably 17.5±14.0 mg, more preferably 17.5±12.0 mg, still more preferably 17.5±10.0 mg, yet more preferably 17.5±8.0 mg, even more preferably 17.5±6.0 mg, most preferably 17.5±4.0 mg, and in particular 17.5±2.0 mg.

Preferably, the weight concentration of the surfactant, preferably TPGS, is at least 0.4 mg/ml; preferably at least 0.8 mg/ml, more preferably at least 1.2 mg/ml, still more preferably at least 1.6 mg/ml, yet more preferably at least 2.0 mg/ml, even more preferably at least 2.4 mg/ml, most preferably at least 2.8 mg/ml, and in particular at least 3.2 mg/ml.

Preferably, the weight concentration of the surfactant, preferably TPGS, is at most 20.0 mg/ml; preferably at most 17.5 mg/ml, more preferably at most 15.0 mg/ml, still more preferably at most 12.5 mg/ml, yet more preferably at most 10.0 mg/ml, even more preferably at most 7.5 mg/ml, most preferably at most 5.0 mg/ml, and in particular at most 4.0 mg/ml.

Preferably, the weight concentration of the surfactant, preferably TPGS, is within the range of 3.5±3.2 mg/ml; preferably 3.5±2.8 mg/ml, more preferably 3.5±2.4 mg/ml, still more preferably 3.5±2.0 mg/ml, yet more preferably 3.5±1.6 mg/ml, even more preferably 3.5±1.2 mg/ml, most preferably 3.5±0.8 mg/ml, and in particular 3.5±0.4 mg/ml.

Preferably, the pharmaceutical dosage form according to the invention contains a buffer (pH buffer).

Preferably, the conjugate acid of the buffer has a pK_{A} value at 25° C. within the range of 8.1±0.8; preferably 8.1±0.7, more preferably 8.1±0.6, still more preferably 8.1±0.5, yet more preferably 8.1±0.4, even more preferably 8.1±0.3, most preferably 8.1±0.2, and in particular 8.1±0.1.

Preferably, the buffer comprises an aliphatic primary amine.

Preferably, the buffer comprises tris(hydroxymethyl)aminomethane (trometamol).

Preferably, the buffer comprises or essentially consists of a combination of tris(hydroxymethyl)aminomethane hydrochloride and tris(hydroxymethyl)aminomethane.

Preferably, the amount of the buffer, preferably trometamol, is at least 0.7 mg; preferably at least 1.4 mg, more preferably at least 2.1 mg, still more preferably at least 2.8 mg, yet more preferably at least 3.5 mg, even more preferably at least 4.2 mg, most preferably at least 4.9 mg, and in particular at least 5.6 mg; preferably as equivalent weight of the conjugate base of the buffer.

Preferably, the amount of the buffer, preferably trometamol, is at most 10.0 mg; preferably at most 9.5 mg, more preferably at most 9.0 mg, still more preferably at most 8.5 mg, yet more preferably at most 8.0 mg, even more preferably at most 7.5 mg, most preferably at most 7.0 mg, and in particular at most 6.5 mg; preferably as equivalent weight of the conjugate base of the buffer.

Preferably, the amount of the buffer, preferably trometamol, is within the range of 6.0±5.6 mg; preferably 6.0±4.9 mg, more preferably 6.0±4.2 mg, still more preferably 6.0±3.5 mg, yet more preferably 6.0±2.8 mg, even more preferably 6.0±2.1 mg, most preferably 6.0±1.4 mg, and in particular 6.0±0.7 mg; preferably as equivalent weight of the conjugate base of the buffer.

Preferably, the molar concentration of the buffer, preferably trometamol, is at least 2.0 mmol/l; preferably at least 3.0 mmol/l, more preferably at least 4.0 mmol/l, still more preferably at least 5.0 mmol/l, yet more preferably at least 6.0 mmol/l, even more preferably at least 7.0 mmol/l, most preferably at least 8.0 mmol/l, and in particular at least 9.0 mmol/l.

Preferably, the molar concentration of the buffer, preferably trometamol, is at most 18 mmol/l; preferably at most 17 mmol/l, more preferably at most 16 mmol/l, still more preferably at most 15 mmol/l, yet more preferably at most 14 mmol/l, even more preferably at most 13 mmol/l, most preferably at most 12 mmol/l, and in particular at most 11 mmol/l.

Preferably, the molar concentration of the buffer, preferably trometamol, is within the range of 10±8 mmol/l; preferably 10±7 mmol/l, more preferably 10±6 mmol/l, still more preferably 10±5 mmol/l, yet more preferably 10±4 mmol/l, even more preferably 10±3 mmol/l, most preferably 10±2 mmol/l, and in particular 10±1 mmol/l.

Preferably, the dosage form according to the invention has a pH value within the range of 8.0±1.6; preferably 8.0±1.4, more preferably 8.0±1.2, still more preferably 8.0±1.0, yet more preferably 8.0±0.8, even more preferably 8.0±0.6, most preferably 8.0±0.4, and in particular 8.0±0.2.

Preferably, the pharmaceutical dosage form according to the invention contains one or more inorganic salts.

Preferably, the one or more inorganic salts comprise or essentially consist of inorganic salts of alkali metals and/or alkaline earth metals.

Preferably, the one or more inorganic salts comprise or essentially consist of chlorides of alkali metals and/or alkaline earth metals.

Preferably, the one or more inorganic salts comprise or essentially consist of NaCl, KCl, CaCl₂), or a combination thereof; preferably a combination of NaCl, KCl, and CaCl₂).

Preferably, the dosage form according to the invention is essentially free of any inorganic salts other than NaCl, KCl and CaCl₂).

Preferably, the total amount of the one or more inorganic salts, preferably NaCl, KCl and CaCl₂), is at least 4.0 mg; preferably at least 10 mg, more preferably at least 16 mg, still more preferably at least 22 mg, yet more preferably at least 28 mg, even more preferably at least 34 mg, most preferably at least 40 mg, and in particular at least 46 mg.

Preferably, the total amount of the one or more inorganic salts, preferably NaCl, KCl and CaCl₂), is at most 90 mg; preferably at most 85 mg, more preferably at most 80 mg, still more preferably at most 75 mg, yet more preferably at most 70 mg, even more preferably at most 65 mg, most preferably at most 60 mg, and in particular at most 55 mg.

Preferably, the total amount of the one or more inorganic salts, preferably NaCl, KCl and CaCl₂), is within the range of 50±48 mg; preferably 50±42 mg, more preferably 50±36 mg, still more preferably 50±30 mg, yet more preferably 50±24 mg, even more preferably 50±18 mg, most preferably 50±12 mg, and in particular 50±6 mg.

Preferably, the total molar concentration of the one or more inorganic salts, preferably NaCl, KCl and CaCl₂), is at least 10 mmol/l; preferably at least 30 mmol/l, more preferably at least 50 mmol/l, still more preferably at least 70 mmol/l, yet more preferably at least 90 mmol/l, even more preferably at least 110 mmol/l, most preferably at least 130 mmol/l, and in particular at least 150 mmol/l.

Preferably, the total molar concentration of the one or more inorganic salts, preferably NaCl, KCl and CaCl₂), is at most 300 mmol/l; preferably at most 280 mmol/l, more preferably at most 260 mmol/l, still more preferably at most 240 mmol/l, yet more preferably at most 220 mmol/l, even more preferably at most 200 mmol/l, most preferably at most 180 mmol/l, and in particular at most 160 mmol/l.

Preferably, the total molar concentration of the one or more inorganic salts, preferably NaCl, KCl and CaCl₂), is within the range of 155±150 mmol/l; preferably 155±130 mmol/l, more preferably 155±110 mmol/l, still more preferably 155±90 mmol/l, yet more preferably 155±70 mmol/l, even more preferably 155±50 mmol/l, most preferably 155±30 mmol/l, and in particular 155±10 mmol/l.

Preferably, the pharmaceutical dosage form according to the invention contains NaCl (NaCl).

Preferably, the one or more inorganic salts comprise NaCl and the amount of the NaCl is at least 5.0 mg; preferably at least 10 mg, more preferably at least 15 mg, still more preferably at least 20 mg, yet more preferably at least 25 mg, even more preferably at least 30 mg, most preferably at least 35 mg, and in particular at least 40 mg.

Preferably, the one or more inorganic salts comprise NaCl and the amount of the NaCl is at most 225 mg; preferably at most 200 mg, more preferably at most 175 mg, still more preferably at most 150 mg, yet more preferably at most 125 mg, even more preferably at most 100 mg, most preferably at most 75 mg, and in particular at most 50 mg.

Preferably, the one or more inorganic salts comprise NaCl and the amount of the NaCl is within the range of 45±40 mg; preferably 45±35 mg, more preferably 45±30 mg, still more preferably 45±25 mg, yet more preferably 45±20 mg, even more preferably 45±15 mg, most preferably 45±10 mg, and in particular 45±5.0 mg.

Preferably, the one or more inorganic salts comprise NaCl and the molar concentration of the NaCl is at least 10 mmol/l; preferably at least 20 mmol/l, more preferably at least 40 mmol/l, still more preferably at least 60 mmol/l, yet more preferably at least 80 mmol/l, even more preferably at least 100 mmol/l, most preferably at least 120 mmol/l, and in particular at least 140 mmol/l.

Preferably, the one or more inorganic salts comprise NaCl and the molar concentration of the NaCl is at most 325 mmol/l; preferably at most 300 mmol/l, more preferably at most 275 mmol/l, still more preferably at most 250 mmol/l, yet more preferably at most 225 mmol/l, even more preferably at most 200 mmol/l, most preferably at most 175 mmol/l, and in particular at most 150 mmol/l.

Preferably, the one or more inorganic salts comprise NaCl and the molar concentration of the NaCl is within the range of 150±140 mmol/l; preferably 150±130 mmol/l, more preferably 150±110 mmol/l, still more preferably 150±90 mmol/l, yet more preferably 150±70 mmol/l, even more preferably 150±50 mmol/l, most preferably 150±30 mmol/l, and in particular 150±10 mmol/l.

Preferably, the pharmaceutical dosage form according to the invention contains KCl (KCl).

Preferably, the one or more inorganic salts comprise KCl and the amount of the KCl is at least 0.1 mg; preferably at least 0.2 mg, more preferably at least 0.4 mg, still more preferably at least 0.6 mg, yet more preferably at least 0.8 mg, even more preferably at least 1.0 mg, most preferably at least 1.2 mg, and in particular at least 1.4 mg.

Preferably, the one or more inorganic salts comprise KCl and the amount of the KCl is at most 5.0 mg; preferably at most 4.5 mg, more preferably at most 4.0 mg, still more preferably at most 3.5 mg, yet more preferably at most 3.0 mg, even more preferably at most 2.5 mg, most preferably at most 2.0 mg, and in particular at most 1.5 mg.

Preferably, the one or more inorganic salts comprise KCl and the amount of the KCl is within the range of 1.5±1.4 mg; preferably 1.5±1.3 mg, more preferably 1.5±1.2 mg, still more preferably 1.5±1.0 mg, yet more preferably 1.5±0.8 mg, even more preferably 1.5±0.6 mg, most preferably 1.5±0.4 mg, and in particular 1.5±0.2 mg.

Preferably, the one or more inorganic salts comprise KCl and the molar concentration of the KCl is at least 0.1 mmol/l; preferably at least 0.5 mmol/l, more preferably at least 1.0 mmol/l, still more preferably at least 1.5 mmol/l, yet more preferably at least 2.0 mmol/l, even more preferably at least 2.5 mmol/l, most preferably at least 3.0 mmol/l, and in particular at least 3.5 mmol/l.

Preferably, the one or more inorganic salts comprise KCl and the molar concentration of the KCl is at most 22.5 mmol/l; preferably at most 20.0 mmol/l, more preferably at most 17.5 mmol/l, still more preferably at most 15.0 mmol/l, yet more preferably at most 12.5 mmol/l, even more preferably at most 10 mmol/l, most preferably at most 7.5 mmol/l, and in particular at most 5.0 mmol/l.

Preferably, the one or more inorganic salts comprise KCl and the molar concentration of the KCl is within the range of 4.0±3.9 mmol/l; preferably 4.0±3.5 mmol/l, more preferably 4.0±3.0 mmol/l, still more preferably 4.0±2.5 mmol/l, yet more preferably 4.0±2.0 mmol/l, even more preferably 4.0±1.5 mmol/l, most preferably 4.0±1.0 mmol/l, and in particular 4.0±0.5 mmol/l.

Preferably, the pharmaceutical dosage form according to the invention contains CaCl₂) (CaCl₂). Preferably, the CaCl₂) used for manufacturing the dosage form is the dihydrate (CaCl₂)₂·H₂O).

Preferably, the one or more inorganic salts comprise CaCl₂) and the amount of the CaCl₂) is at least 0.2 mg; preferably at least 0.4 mg, more preferably at least 0.8 mg, still more preferably at least 1.2 mg, yet more preferably at least 1.6 mg, even more preferably at least 2.0 mg, most preferably at least 2.4 mg, and in particular at least 2.8 mg; preferably as equivalent weight of CaCl₂) dihydrate.

Preferably, the one or more inorganic salts comprise CaCl₂) and the amount of the CaCl₂) is at most 10 mg; preferably at most 9.0 mg, more preferably at most 8.0 mg, still more preferably at most 7.0 mg, yet more preferably at most 6.0 mg, even more preferably at most 5.0 mg, most preferably at most 4.0 mg, and in particular at most 3.0 mg; preferably as equivalent weight of CaCl₂) dihydrate.

Preferably, the one or more inorganic salts comprise CaCl₂) and the amount of the CaCl₂) is within the range of 3.0±2.8 mg; preferably 3.0±2.6 mg, more preferably 3.0±2.4 mg, still more preferably 3.0±2.0 mg, yet more preferably 3.0±1.6 mg, even more preferably 3.0±1.2 mg, most preferably 3.0±0.8 mg, and in particular 3.0±0.4 mg; preferably as equivalent weight of CaCl₂) dihydrate.

Preferably, the one or more inorganic salts comprise CaCl₂) and the molar concentration of the CaCl₂) is at least 0.1 mmol/l; preferably at least 0.5 mmol/l, more preferably at least 1.0 mmol/l, still more preferably at least 1.5 mmol/l, yet more preferably at least 2.0 mmol/l, even more preferably at least 2.5 mmol/l, most preferably at least 3.0 mmol/l, and in particular at least 3.5 mmol/l.

Preferably, the one or more inorganic salts comprise CaCl₂) and the molar concentration of the CaCl₂) is at most 22.5 mmol/l; preferably at most 20.0 mmol/l, more preferably at most 17.5 mmol/l, still more preferably at most 15.0 mmol/l, yet more preferably at most 12.5 mmol/l, even more preferably at most 10 mmol/l, most preferably at most 7.5 mmol/l, and in particular at most 5.0 mmol/l.

Preferably, the one or more inorganic salts comprise CaCl₂) and the molar concentration of the CaCl₂) is within the range of 4.0±3.9 mmol/l; preferably 4.0±3.5 mmol/l, more preferably 4.0±3.0 mmol/l, still more preferably 4.0±2.5 mmol/l, yet more preferably 4.0±2.0 mmol/l, even more preferably 4.0±1.5 mmol/l, most preferably 4.0±1.0 mmol/l, and in particular 4.0±0.5 mmol/l.

In preferred embodiments, the dosage form according to the invention additionally contains one or more bulking agents, wherein the one or more bulking agents comprise or essentially consist of (a) a monosaccharide; preferably dextrose; (b) a disaccharide; preferably, sucrose, lactose, trehalose, or a combination thereof; (c) a sugar alcohol; preferably mannitol, sorbitol, or a combination thereof; (d) a polysaccharide; preferably dextran, cyclodextrin, or a combination thereof; (e) an amino acid; preferably glycine, alanine, proline, arginine, histidine, or a combination thereof; or (f) a synthetic polymer; preferably PVP, PEG, or a combination thereof; or a combination of any of (a) through (f).

Preferably, the one or more bulking agents comprise or essentially consist of (c) a sugar alcohol; preferably mannitol, sorbitol, or a combination thereof; more preferably mannitol.

In other preferred embodiments, the dosage form according to the invention does not contain a bulking agent.

Another aspect of the invention relates to a kit for the preparation of a dosage form according to the invention as described above by combining a first component and a second component with one another; wherein said first component contains the total amount of the Resiniferatoxin; wherein said second component contains the total amount of the water; and wherein the organic solvent, the surfactant, the optional buffer, and the optional one or more inorganic salts, independently of one another are contained either only in said first component, or only in said second component, or in both said first component as well as said second component.

In preferred embodiment, said first component containing the total amount of the Resiniferatoxin is a solid, preferably a lyophilizate.

In other preferred embodiment, said first component containing the total amount of the Resiniferatoxin is a liquid, preferably a solution, more preferably an ethanolic solution.

Another aspect of the invention relates to the dosage form according to the invention as described above or the kit according to the invention as described above for use in the treatment of knee joint pain.

Another aspect of the invention relates to a method of treating knee joint pain comprising administering the dosage form according to the invention as described above to a subject in need thereof.

A further aspect of the invention relates to the use of Resiniferatoxin for the preparation of the dosage form according to the invention as described above for the treatment of knee joint pain.

Preferably, the knee joint pain is osteoarthritic knee joint pain (knee joint pain due to osteoarthritis). Thus, the pain to be treated is preferably associated to osteoarthritis of the knee.

Preferably, the pain is moderate or severe.

In preferred embodiments, the knee joint pain is osteoarthritic knee joint pain associated with osteoarthritis of Kellgren-Lawrence Grade 2 (minimal).

In preferred embodiments, the knee joint pain is osteoarthritic knee joint pain associated with osteoarthritis of Kellgren-Lawrence Grade 3 (moderate).

In preferred embodiments, the knee joint pain is osteoarthritic knee joint pain associated with osteoarthritis of Kellgren-Lawrence Grade 4 (severe).

In preferred embodiments, the knee joint pain is osteoarthritic knee joint pain associated with osteoarthritis of Ahlback Grade 1 (joint space <3 mm).

In preferred embodiments, the knee joint pain is osteoarthritic knee joint pain associated with osteoarthritis of Ahlback Grade 2 (joint space obliteration).

In preferred embodiments, the knee joint pain is osteoarthritic knee joint pain associated with osteoarthritis of Ahlback Grade 3 (minor bone attrition, 0-5 mm).

In preferred embodiments, the knee joint pain is osteoarthritic knee joint pain associated with osteoarthritis of Ahlback Grade 4 (moderate bone attrition. >5-10 mm).

In preferred embodiments, the knee joint pain is osteoarthritic knee joint pain associated with osteoarthritis of Ahlback Grade 5 (severe bone attrition, >10 mm).

The Ahlback and the Kellgren-Lawrence grading systems are known to the skilled person (S Ahlback, Osteoarthrosis of the knee: a radiographic investigation. Acta Radiol Suppl 1968, 277:7-72; J H Kellgren, J S Lawrence, Radiologic assessment of osteoarthritis. Ann Rheum Dis 1957 16:494-501) and can be compared to one another as follows:

| Ahlbäck | | Kellgren-Lawrence | |
|---|---|---|---|
| grade | definition | grade | definition |
| — | — | 1 | doubtful; minute osteophytes, |
| | | | doubtful significance |
| — | — | 2 | minimal; definite osteophytes, |
| | | | unimpaired joint space |
| 1 | joint space narrowing | 3 | moderate; moderate diminution |
| | (joint space <3 mm) | | of joint space |
| 2 | joint space obliteration | 4 | severe; joint space greatly |
| | | | of impaired with sclerosis |
| | | | subchondral bone |
| 3 | minor bone attrition | 4 | as above |
| | (0-5 mm) | | |
| 4 | moderate bone attrition | 4 | as above |
| | (>5-10 mm) | | |
| 5 | severe bone attrition | 4 | as above |
| | (<10 mm) | | |

Preferably, the pain is chronic.

Unless expressly stated otherwise, the osteoarthritis of the knee is in accordance with the International Classification of Diseases, ICD-11 (version 1/2023), FA01.

Osteoarthritis of the knee is described therein as primary osteoarthritis occurring in an otherwise intact knee joint, involving genetically related, age-related or use-related degeneration with microscopic and macroscopic anatomical changes, which ultimately limit motion in one or more joints. Changes to the joint include increasing cartilage loss and osseous transformation such as sclerosis, osteophyte formation and cysts as well as potential inflammatory changes in surrounding soft tissue structures.

In preferred embodiments, the osteoarthritis of the knee is bilateral (XK9J), left (XK8G), right (XK9K), or unilateral unspecified (XK70).

In preferred embodiments, the pain that is associated to the osteoarthritis of the knee, i.e. the osteoarthritic knee joint pain, is selected from chronic post traumatic pain (MG30.20) and chronic secondary musculoskeletal pain associated with structural changes (MG30.31).

Within chronic post traumatic pain (MG30.20) or chronic secondary musculoskeletal pain associated with structural changes (MG30.31), the pain may have severity selected from mild pain (XS5D), moderate pain (XS9Q), and severe pain (XS2E). Within chronic post traumatic pain (MG30.20) or chronic secondary musculoskeletal pain associated with structural changes (MG30.31), the pain may have alternative severity 1 selected from mild distress (XS3R), moderate distress (XS7C), and severe distress (XS7N). Within chronic post traumatic pain (MG30.20) or chronic secondary musculoskeletal pain associated with structural changes (MG30.31), the pain may have alternative severity 2 selected from mild pain-related interference (XS5R), moderate pain-related interference (XS2L), and severe pain-related interference (XS2U). Within chronic post traumatic pain (MG30.20) or chronic secondary musculoskeletal pain associated with structural changes (MG30.31), the pain may have temporal pattern and onset selected from intermittent (XT5G), persistent (XT6Z), and persistent with overlaid attacks (XT5T).

Preferably, the dosage form is administered by intraarticular administration (IA).

Preferably, intraarticular administration is intraarticular injection.

Thus, upon intraarticular administration the dosage form remains within the joint, typically within the joint fluid; no perfusion or infusion is performed.

For intraarticular injection the patient is preferably placed in the supine position. A small pillow or towel is preferably placed under the knee thereby producing slight flexion to the hip and knee. This results in relaxation of the extensor muscles. While it is contemplated that injection may be performed via the lateral route, the medial route or the anterior route, the lateral route is preferred. After sterile preparation and draping of the skin along the lateral aspect of the patella, the patella is preferably pushed slightly laterally to open the joint. The sift tissues just inferior (posterior) to the lateral patella and its midportion can be anesthetized. For example, a 22 gauge (0.644 mm) needle may be directed anteriorly and cephalad (at about 45 degrees in each direction) until the patella is contacted. Aspiration of joint fluid is then preferably attempted.

The total volume of the pharmaceutical dosage form according to the invention is administered, preferably injected, into the knee joint within a few seconds or minutes. After administration is complete, the injection needle is removed.

Intraarticular administration may be monolateral or bilateral.

Preferably, the patient is a human, preferably from 35 to 85 years of age.

In preferred embodiments a patient is treated who has insufficient pain relief on optimal standard of care. In preferred embodiments a patient is treated who is unable to be on optimal standard of care due to contraindication or intolerability. In this regard, optimal standard of care includes one or more of non-pharmacological treatment, oral or topical treatment with NSAIDS, oral treatment with opioids, intraarticular treatment with corticosteroids, and intraarticular treatment with hyaluronic acid.

In preferred embodiments a patient is treated who prior to intraarticular administration has not been locally anesthetized at the site of intraarticular administration.

In other preferred embodiments a patient is treated who prior to intraarticular administration has been locally anesthetized at the site of intraarticular administration, preferably with Ropivacaine, Bupivacaine or Lidocaine, more preferably Ropivacaine.

In preferred embodiments, the treatment comprises intraarticular administration, preferably intraarticular injection, of a first liquid pharmaceutical dosage form according to the invention on a first day of administration and of a second liquid pharmaceutical dosage form according to the invention on a second day of administration; wherein the second day of administration is at least 1 month after the first day of administration; and wherein no Resiniferatoxin is administered between the first day of administration and the second day of administration.

Preferably, the dose of Resiniferatoxin in the first liquid pharmaceutical dosage form is within the range of 400±200 ng; preferably 400±175 ng, more preferably 400±150 ng, still more preferably 400±125 ng, yet more preferably 400±100 ng, even more preferably 400±75 ng, most preferably 400±50 ng, and in particular 400±25 ng.

Preferably, the dose of Resiniferatoxin in the second liquid pharmaceutical dosage form is within the range of 400±200 ng; preferably 400±175 ng, more preferably 400±150 ng, still more preferably 400±125 ng, yet more preferably 400±100 ng, even more preferably 400±75 ng, most preferably 400±50 ng, and in particular 400±25 ng.

Preferably, the dose of Resiniferatoxin in the first liquid pharmaceutical dosage form is the same as the dose of Resiniferatoxin in the second liquid pharmaceutical dosage form.

Preferably, the second day of administration is at least 2 months, preferably at least 3 months, more preferably at least 4 months, still more preferably at least 5 months, yet more preferably at least 6 months after the first day of administration.

In preferred embodiments, the second day of administration is determined according to the individual subjective pain perception of the patient.

In preferred embodiments, the treatment further comprises intraarticular administration, preferably intraarticular injection, of a third liquid pharmaceutical dosage form according to the invention on a third day of administration; wherein the third day of administration is at least 1 month after the second day of administration; and wherein no Resiniferatoxin is administered between the second day of administration and the third day of administration.

Preferably, the dose of Resiniferatoxin in the third liquid pharmaceutical dosage form is within the range of 400±200 ng; preferably 400±175 ng, more preferably 400±150 ng, still more preferably 400±125 ng, yet more preferably 400±100 ng, even more preferably 400±75 ng, most preferably 400±50 ng, and in particular 400±25 ng.

Preferably, the dose of Resiniferatoxin in the second liquid pharmaceutical dosage form is the same as the dose of Resiniferatoxin in the third liquid pharmaceutical dosage form.

Preferably, the third day of administration is at least 2 months, preferably at least 3 months, more preferably at least 4 months, still more preferably at least 5 months, yet more preferably at least 6 months after the second day of administration.

In preferred embodiments, the treatment comprises intraarticular administration, preferably intraarticular injection, of liquid pharmaceutical dosage forms according to the invention every second year, one time per year, two times per year, three times per year, four times per year, five times per year, or six times per year, and wherein the total duration of treatment is at least 1 year.

Preferably, each of the liquid pharmaceutical dosage forms comprises Resiniferatoxin at a dose within the range of 400±200 ng; preferably 400±175 ng, more preferably 400±150 ng, still more preferably 400±125 ng, yet more preferably 400±100 ng, even more preferably 400±75 ng, most preferably 400±50 ng, and in particular 400±25 ng.

Preferably, the dose of Resiniferatoxin in each of the liquid pharmaceutical dosage forms is the same.

Preferably, the total duration of treatment is at least 2 years, preferably at least 3 years, more preferably at least 4 years.

The following examples further illustrate the invention but are not to be construed as limiting its scope:

### Example 1—Study in Humans

In a human study, two pharmaceutical dosage forms according to the invention were used comprising Resiniferatoxin (100 or 400 ng) in an aqueous ethanolic solution additionally containing polysorbate 80 (Tween® 80) as surfactant, trometamol as buffer and a combination of NaCl, KCl, and CaCl₂) as inorganic salts.

The primary objective of this study was to compare the analgesic effects of single intraarticular injections of Resiniferatoxin (100 or 400 ng) compared to placebo, at 3 and 6 months postinjection, in participants with chronic osteoarthritic knee joint pain. Secondary objectives were to evaluate the analgesic effects of Resiniferatoxin versus placebo in terms of a responder analysis (percentage of subjects achieving ≥50% or ≥70% reduction in visual analog scale pain score) and changes in Western Ontario and McMaster Universities Arthritis Index (WOMAC) total and subscale scores (pain, physical function and stiffness). Safety and injection site pain were also evaluated.

The study was conducted as a randomized, double-blind, placebo-controlled, single-dose trial of Resiniferatoxin. Eligible patients were aged 40 to 80 years with radiographic knee osteoarthritis (Kellgren-Lawrence Grade 2-4 in the last 3 years) and a baseline visual analog scale (0-100 mm) pain score ≥40 mm on motion in the target knee, with or without pain medication. 67 subjects were randomized to treatment with either Resiniferatoxin 100 ng (n=24) or 400 ng (n=23), or placebo (n=20), administered intraarticularly into the index knee. Intraarticular Ropivacaine (5 ml, 0.5%) was administered 15 mins before investigational medicinal product. Visual analog scale scores were used to evaluate pain on motion as the average of the last 2 days in a target knee between baseline and at 3 and 6 months post injection. Injection site pain was rated (visual analog scale) from baseline (−0.5 h) up to 3 h post intraarticular injections.

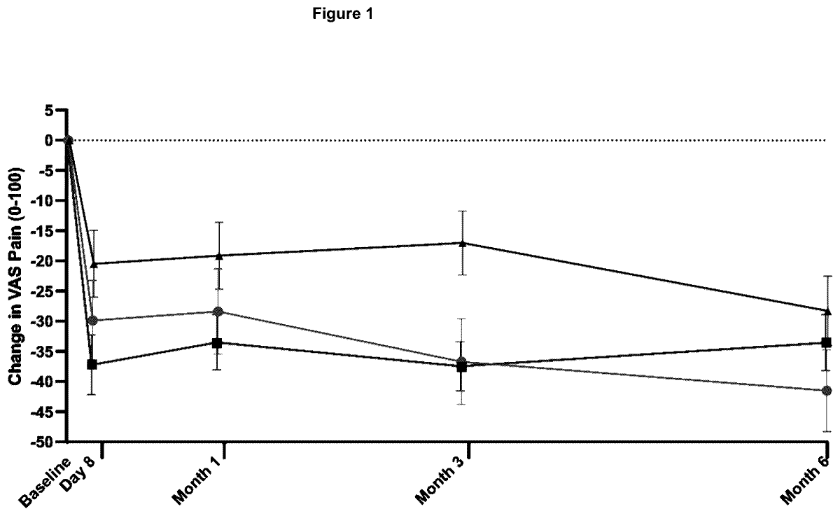

Baseline demographic characteristics and osteoarthritis pain scores were comparable across treatment groups. In the intention-to-treat population, a reduction in the visual analog scale scores for pain on motion in the treated knee after intraarticular injection was observed as early as the first trial visit post-injection (day 8) with the effect lasting until the last trial visit (month 6; FIG. 1). Subjects receiving Resiniferatoxin 100 ng or Resiniferatoxin 400 ng reported a greater pain reduction than subjects receiving placebo. At the 3 months visit, there was a higher mean [SD] absolute reduction of visual analog scale score (baseline corrected) in the Resiniferatoxin 100 ng group (37.43 [19.79]) and the Resiniferatoxin 400 ng group (36.68 [34.16]) than in the placebo group (17.00 [23.09]). At 6 months, the mean (SD) absolute reduction of visual analog scale score was 33.52 (22.89) in the Resiniferatoxin 100 ng group, 41.48 (32.57) in the Resiniferatoxin 400 ng group, and 28.26 (25.02) in the placebo group. Consistent with these data, the Resiniferatoxin 100 ng and Resiniferatoxin 400 ng treatment groups showed a greater reduction in WOMAC total and subscale scores (pain, physical function and stiffness) than the placebo group at both 3 and 6 months.

The incidence of treatment emergent adverse events in the Resiniferatoxin 400 ng group (78.3%) and placebo group (75.0%) were comparable, but lower in the Resiniferatoxin 100 ng group (62.5%). The most commonly reported treatment emergent adverse events were arthralgia, back pain, nasopharyngitis and headache. The majority of treatment emergent adverse events were mild and unrelated to investigational medicinal product or Ropivacaine. Seven severe adverse events were reported in 6 subjects [2 subjects (Resiniferatoxin 100 ng), 3 subjects (Resiniferatoxin 400 ng) 1 subject (placebo)], none of which were considered to be related to the investigational medicinal product or Ropivacaine. No safety concerns were raised based on other evaluated safety parameters. Injection site pain (procedural pain) was highly variable across treatment groups, although generally higher following intraarticular Resiniferatoxin compared to placebo, with peak pain (moderate-severe intensity in some subjects) noted at around 0.5 h post-injection and return to baseline by 3h.

The data from this exploratory trial indicate that single intraarticular doses of Resiniferatoxin (100 ng or 400 ng) has the potential to mediate clinically-meaningful pain relief in an osteoarthritic knee population. Analgesic onset occurred within one week of administration and was evident for at least 3 months during the follow-up period. Injection site pain was a very common event. Resiniferatoxin was found to have a good safety profile and to have been well tolerated.

FIG. 1 shows the efficacy in terms of visual analog scale pain in motion, change over time, 2-day recall, intention-to-treat population; n=number of subjects with data; VAS=visual analogue scale. Data values plotted as mean±standard error of mean, offset in X for clarity. ▴ Placebo (n=20); ▪ Resiniferatoxin 100 ng (n=24); ● Resiniferatoxin 400 ng (n=23).

### Example 2—Comparative Study in Rats

The primary objective of this study was an investigation of pharmacokinetic similarity of Resiniferatoxin (RTX) in plasma, synovial fluid (SF) and knee tissues (meniscus, patella, femoral head and tibial plateau) in male rats (n=3 per group) after intraarticular (IA) dosing of 9.6 ng/knee RTX using 3 formulations (inventive example I-1=ethanolic liquid, comparative example C-1=freeze dried without bulking agent, and comparative example C-2=freeze dried with mannitol):

| mg per volume of 5 mL | I-1 | C-1 | C-2 |
|---|---|---|---|
| Resiniferatoxin (RTX) | 0.0016 | 0.0016 | 0.0016 |
| ethanol abs. | 197.2 | — | — |
| TPGS | 17.5 | 17.5 | 17.5 |
| mannitol | — | — | 74.5 |
| NaCl | 42.9 | 45 | 45 |
| CaCl₂•2H₂O | 2.9 | 1.0 | 2.9 |
| KCl | 1.5 | 0.5 | 1.5 |
| trometamol (TRIS) | 6.1 | 2.0 | 6.1 |
| HCl | q.s. pH 8 | q.s. pH 8 | q.s. pH 8 |
| water for injection | 4778 | 4778 | 4778 |

Animals: 63 male rats (21 rats per formulation; plus an additional 3 animals as a replacement). Body weight: average 263 g (236-295 g). Dose (IA): ˜36.6 ng/kg=>9.6 ng/knee as an injection of 30 μL/knee of three various formulations with concentration 320 ng/mL. PK sampling: 7 timepoints: 10 min (animals not fully conscious between dosing and sampling), 20 min, 30 min, 1 h, 1.5 h, 2 h and 4 h (one sample per rat; 3 rats per timepoint) in plasma and synovial fluid. Tissue samples: at same time points as PK samples following tissue samples: meniscus, patella, femoral head and tibial plateau.

### Results:

### a) Tissue Concentration Data:

| inventive | tₘₐₓ | Cₘₐₓ | SE_Cₘₐₓ | AUC_{0-4 h} | SE_AUC_{0-4 h} | tₗₐₛₜ | t_{1/2, z} |
|---|---|---|---|---|---|---|---|
| example I-1 | (h) | (pg/g) | (pg/g) | (h*pg/g) | (h*pg/g) | (h) | (h) |
| Meniscus | 1.00 | 28870 | 4813 | 40630 | 3048 | 4.00 | 1.32 |
| Patella | 0.33 | 5053 | 939 | 8421 | 899 | 4.00 | 2.57 |
| Femoral | 0.17 | 7160 | 500 | 11760 | 639 | 4.00 | 0.895 |
| head | | | | | | | |
| Tibial | 0.33 | 8107 | 1627 | 7058 | 477 | 4.00 | 1.29 |
| plateau | | | | | | | |

| comparative | tₘₐₓ | Cₘₐₓ | SE_Cₘₐₓ | AUC_{0-4 h} | SE_AUC_{0-4 h} | tₗₐₛₜ | t_{1/2, z} |
|---|---|---|---|---|---|---|---|
| example C-1 | (h) | (pg/g) | (pg/g) | (h*pg/g) | (h*pg/g) | (h) | (h) |
| Meniscus | 0.17 | 22400 | 6991 | 32500 | 3259 | 4.00 | 1.47 |
| Patella | 0.50 | 4030 | 520 | 7516 | 1042 | 4.00 | 1.77 |
| Femoral | 0.17 | 7883 | 536 | 10080 | 609 | 4.00 | 0.925 |
| head | | | | | | | |
| Tibial | 0.17 | 14620 | 4164 | 8079 | 1216 | 4.00 | 1.09 |
| plateau | | | | | | | |

| | |
|---|---|
| | Deviation to |
| comparative | inventive example I-1 in % |

| example C-1 | Cₘₐₓ | AUC_{0-4 h} |
|---|---|---|
| Meniscus | −22% | −20% |
| Patella | −20% | −11% |
| Femoral head | 10% | −14% |
| Tibial plateau | 80% | 14% |

| comparative | tₘₐₓ | Cₘₐₓ | SE_Cₘₐₓ | AUC_{0-4 h} | SE_AUC_{0-4 h} | tₗₐₛₜ | t_{1/2, z} |
|---|---|---|---|---|---|---|---|
| example C-2 | (h) | (pg/g) | (pg/g) | (h*pg/g) | (h*pg/g) | (h) | (h) |
| Meniscus | 0.50 | 20800 | 5934 | 31640 | 4127 | 4.00 | 3.21 |
| Patella | 0.50 | 6300 | 2300 | 8417 | 1114 | 4.00 | 1.31 |
| Femoral | 0.50 | 8180 | 1030 | 11290 | 613 | 4.00 | 0.916 |
| head | | | | | | | |
| Tibial | 0.17 | 6410 | 904 | 6577 | 565 | 4.00 | 1.17 |
| plateau | | | | | | | |

| | |
|---|---|
| | Deviation to |
| comparative | inventive example I-1 in % |

| example C-2 | Cₘₐₓ | AUC_{0-4 h} |
|---|---|---|
| Meniscus | −28% | −22% |
| Patella | 25% | 0% |
| Femoral head | 14% | −4% |
| Tibial plateau | −21% | −7% |

Highest exposure was found in meniscus followed by femoral head, patella and tibial plateau. RTX had a short half-life of about 1.5 h in the tissues (range: 0.895 h to 3.21 h).

Cₘₐₓ values of comparative example C-1 and comparative example C-2 were closer to inventive example I-1 for femoral head (within 15%), whereas for meniscus, patella and tibial plateau, Cₘₐₓ of comparative example C-1 and comparative example C-2 were 20 to 80% different to inventive example I-1.

AUC values of comparative example C-1 and comparative example C-2 were within 20% of inventive example I-1 for patella, femoral head and tibial plateau and 20-22% different for meniscus.

### b) Synovial Fluid and Plasma Pharmacokinetic Data:

After intraarticular dose of 9.6 ng/knee to male rats, synovial fluid concentrations were measurable up to 4 hours in all three formulations.

Sampling of synovial fluid: A 0.5 ml 28G Insulin syringes (fixed needle, no hub) were filled with 100 μl of the saline/EDTA solution. The skin was removed from the joint and then the needle was inserted through the patellar tendon into the joint space. The solution was then injected into the joint space, the knee was flexed a couple/few times (all with the needle still in the joint) and then was pulled back on the plunger to collected the mix of saline/EDTA and synovial fluid. The lavage fluid was then put into a microcentrifuge tube, it was spun to pellet any cells/debris and then the supernatant was collected.

Inventive example I-1 showed a synovial fluid concentration profile with initially high synovial fluid concentrations (tₘₐₓ=10 min) followed by a constant fast decline until 2 h. Comparative example C-1 and comparative example C-2 showed very similar PK profile synovial fluid concentrations with mean tₘₐₓ at 20 min.

Due to the different initial PK profile in synovial fluid of inventive example I-1 compared to comparative example C-1 and comparative example C-2, the mean Cₘₐₓ was at least ˜5-fold and AUC is ˜3-fold higher for inventive example I-1:

| | I-1 | C-1 | C-2 |
|---|---|---|---|
| C₀ [pg · mL⁻¹] | 182,000 | 3,440 | 2,290 |
| tₘₐₓ [h] | 0.167 | 0.333 | 0.333 |
| Cₘₐₓ [pg · mL⁻¹] | 21,400 | 4,060 | 3,910 |
| AUC₀₋₄ [h · pg · mL⁻¹] | 20,100 | 2,050 | 2,040 |
| AUC_{inf} [h · pg · mL⁻¹] | 20,200 | 2,060 | 2,060 |
| t_{1/2, z} [h] | 1.85 | 1.07 | 1.64 |

Very low plasma concentrations were measurable until 1 h. Similar plasma concentration profiles were found for the different formulations.

In summary, after intraarticular dose of 9.6 ng/knee to male rats, synovial fluid and knee tissue concentrations were measurable up to 4 hours (last timepoint) in all three formulations. Very low plasma concentrations were measurable only until 1 h with all three formulations.

For synovial fluid and plasma: Inventive example I-1 had a synovial fluid concentration-time profile with initial high concentrations (tₘₐₓ=10 min) followed by a fast distribution phase until 2 h. Comparative example C-1 and comparative example C-2 had a synovial fluid concentration-time profile with a later tₘₐₓ at 20 min and lower Cₘₐₓ, which was different from inventive example I-1. Due to this initial difference in the synovial fluid profile of inventive example I-1 compared to comparative example C-1 and comparative example C-2, the mean Cₘₐₓ was at least ˜5-fold and AUC was at least ˜3-fold higher for inventive example I-1. However, the plasma exposure differences from the inventive example I-1 was lower: −21% to −29% for comparative example C-1 and −11% to −14% for comparative example C-2.

For the knee tissues: Cₘₐₓ values of comparative example C-1 and comparative example C-2 were closer to inventive example I-1 for femoral head (within 15%), whereas for meniscus, patella and tibial plateau, Cₘₐₓ of comparative example C-1 and comparative example C-2 were 20 to 80% different to inventive example I-1. AUC values of comparative example C-1 and comparative example C-2 were within 20% of inventive example I-1 for patella, femoral head and tibial plateau and 20-22% different for meniscus.

Highest tissue exposures were found in meniscus followed by femoral head, patella and tibial plateau.

RTX had a short half-life of approximately 1.5 h in SF (range: 1.07 h to 1.85 h) and in the tissues (range: 0.895 h to 3.21 h).

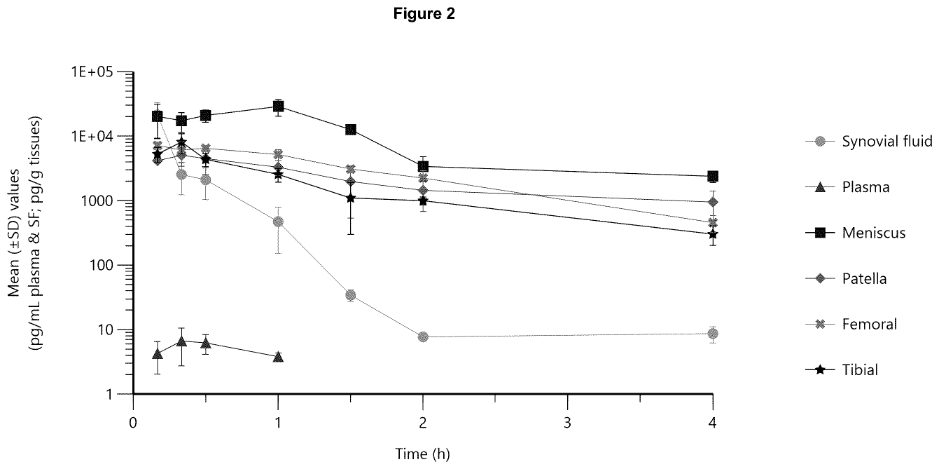

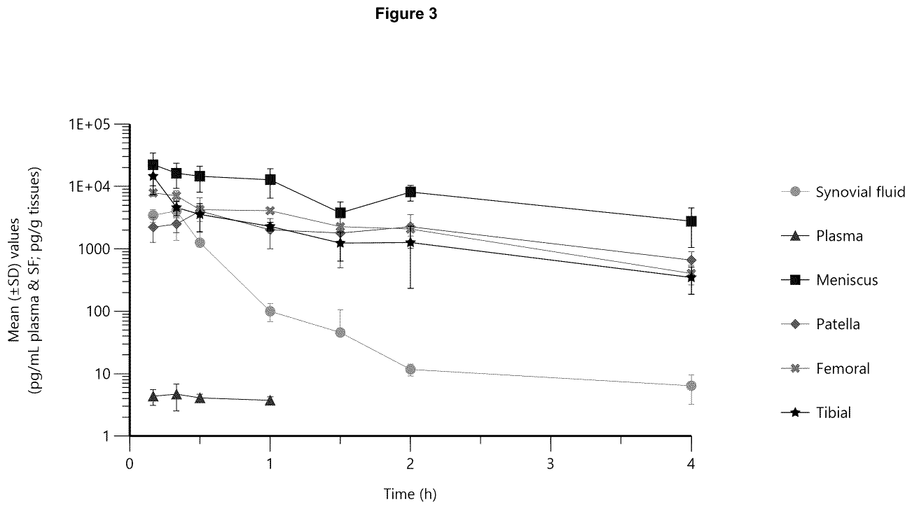

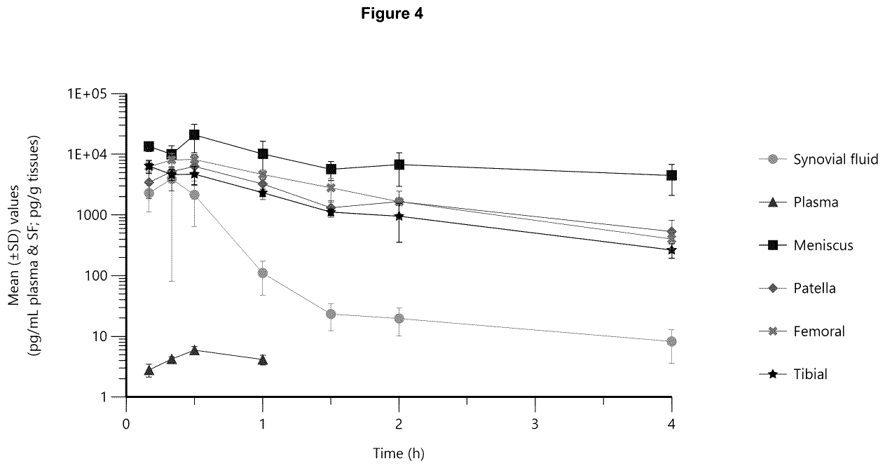

FIGS. 2, 3 and 4 show the PK concentration time plots for inventive example I-1 (FIG. 2), comparative example C-1 (FIG. 3), and comparative example C-2 (FIG. 4), respectively.

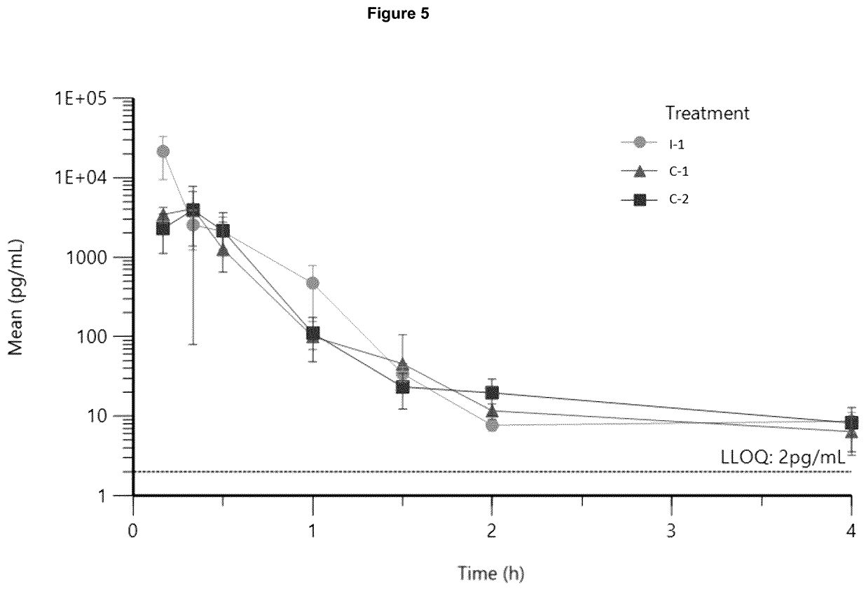

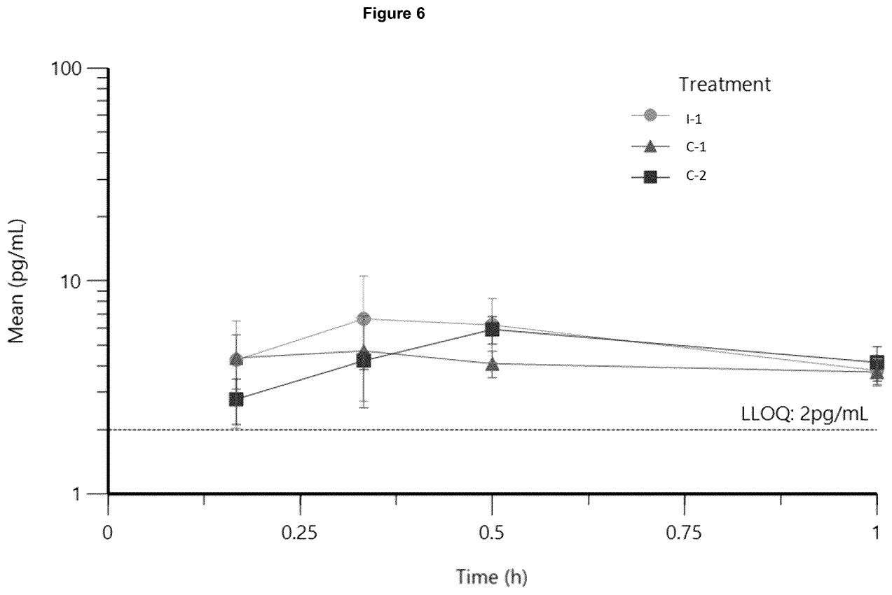

FIGS. 5 and 6 show PK in synovial fluid (FIG. 5) and plasma (FIG. 6), respectively.

It can be concluded that upon intraarticular administration the distribution of Resiniferatoxin within the knee can be improved when the injection formulation contains ethanol.

1. A pharmaceutical dosage form for injection comprising or essentially consisting of
(i) Resiniferatoxin;
(ii water;
(iii) an organic solvent;
(iv) a surfactant;
(v) optionally, a buffer; and
(vi) optionally, one or more inorganic salts;
wherein a weight content of the water is greater than a weight content of the organic solvent.

2. (canceled)

3. The dosage form according to claim 1, wherein the dose of Resiniferatoxin is less than 500 ng.

4. (canceled)

5. (canceled)

6. (canceled)

7. The dosage form according to claim 1, wherein the dose of Resiniferatoxin is within the range of 400±25 ng.

8. (canceled)

9. (canceled)

10. (canceled)

11. The dosage form according to claim 1, which has a total volume of at least 1.0 ml and at most 12 ml.

12. (canceled)

13. (canceled)

14. (canceled)

15. (canceled)

16. (canceled)

17. (canceled)

18. (canceled)

19. The dosage form according to claim 1, wherein a weight content of water is at least 88 wt.-% relative to a total weight of the dosage form.

20. The dosage form according to claim 1, wherein the organic solvent is ethanol or dimethyl sulfoxide.

21. The dosage form according to claim 20, wherein the organic solvent is ethanol.

22. (canceled)

23. (canceled)

24. (canceled)

25. (canceled)

26. (canceled)

27. (canceled)

28. (canceled)

29. (canceled)

30. (canceled)

31. The dosage form according to claim 1, wherein the surfactant is a nonionic surfactant.

32. (canceled)

33. The dosage form according to claim 1, wherein the surfactant is D-α-tocopherol polyethylene glycol succinate (TPGS) selected from TPGS 200, TPGS 238, TPGS 400, TPGS 600, TPGS 1000, TPGS 2000, TPGS 3400, TPGS 3500, TPGS 4000, TPGS 6000, and mixtures thereof.

34. (canceled)

35. The dosage form according to claim 1, wherein the surfactant is selected from polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), and polysorbate 80 (polyoxyethylene (20) sorbitan monooleate).

36. (canceled)

37. (canceled)

38. (canceled)

39. The dosage form according to claim 1, wherein the weight concentration of the surfactant is at least 0.4 mg/ml and at most 20.0 mg/ml.

40. (canceled)

41. (canceled)

42. The dosage form according to claim 1, wherein a conjugate acid of the buffer has a pK_{A} value at 25° C. within the range of 8.1±0.8.

43. (canceled)

44. The dosage form according to claim 1, wherein the buffer comprises tris(hydroxymethyl)aminomethane (trometamol).

45. (canceled)

46. (canceled)

47. (canceled)

48. (canceled)

49. (canceled)

50. (canceled)

51. (canceled)

52. The dosage form according to claim 1, which has a pH value within the range of 8.0±0.6.

53. (canceled)

54. (canceled)

55. The dosage form according to claim 1, wherein the one or more inorganic salts comprise or essentially consist of NaCl, KCl, CaCl₂, or a combination thereof.

56. The dosage form according to claim 1, which is essentially free of any inorganic salts other than NaCl, KCl and CaCl₂.

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. A kit for the preparation of a dosage form according to claim 1 by combining a first component and a second component with one another;
wherein said first component contains the total amount of the Resiniferatoxin;
wherein said second component contains the total amount of the water; and
wherein the organic solvent, the surfactant, the optional buffer, and the optional one or more inorganic salts, independently of one another are contained
either only in said first component,
or only in said second component,
or in both said first component as well as said second component.

86. A method for treating joint pain in a patient in need thereof, said method comprising administering to said patient the dosage form according to claim 1.

87. The method according to claim 86, which is administered to said patient by intraarticular administration.

88. (canceled)

89. The method according to claim 86, wherein the dose of Resiniferatoxin is within the range of 400±25 ng.

90. (canceled)

91. (canceled)

92. (canceled)

93. The method according to claim 86, wherein the dosage form has a liquid volume within the range of 5.00±2.00 ml.

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. The method according to claim 86, wherein the treatment comprises intraarticular administration of a first such liquid pharmaceutical dosage form on a first day of administration and of a second such liquid pharmaceutical dosage form on a second day of administration; wherein the second day of administration is at least 1 month after the first day of administration; and wherein no Resiniferatoxin is administered between the first day of administration and the second day of administration.

115. (canceled)

116. (canceled)

117. (canceled)

118. (canceled)

119. (canceled)

120. (canceled)

121. (canceled)

122. (canceled)

123. (canceled)

124. (canceled)

125. (canceled)

126. (canceled)
